(19) United States
(12) Patent Application Publication
(10) Pub. No.: US 2018/0200297 A1
(43) Pub. Date: Jul. 19, 2018

(54) COMPOSITION COMPRISING CARNITINE, SODIUM CHOLATE, SODIUM ACETATE AND OPTIONALLY SILVER FOR USE IN THE TREATMENT OF PSORIASIS, VITILIGO AND ROSACEA

(71) Applicant: GK PHARMA CONSULTANTS SA, Chiasso (CH)

(72) Inventor: Enrico Magni, Chiasso (CH)

(73) Assignee: GK PHARMA CONSULTANTS SA, Chiasso (CH)

(21) Appl. No.: 15/869,147

(22) Filed: Jan. 12, 2018

(30) Foreign Application Priority Data
Jan. 16, 2017 (IT) 102017000004019

(51) Int. Cl. A61K 33/38; A61K 31/205; A61K 31/575; A61K 31/19; A61P 17/06; A61P 17/00

(52) U.S. Cl. CPC A61K 33/38; A61K 31/205; A61P 17/00; A61K 31/19; A61P 17/06; A61K 31/575

(57) ABSTRACT

Disclosed is a composition comprising:
a) carnitine,
b) sodium cholate,
c) sodium acetate,
   and optionally
d) silver,
   for use in the treatment of psoriasis, vitiligo and rosacea.

This non-provisional application claims priority to and the benefit of Italian Application No. 102017000004019 filed on Jan. 16, 2017 the content of which is incorporated herein by reference in its entirety.

### FIELD OF INVENTION

The present invention relates to a pharmaceutical composition comprising carnitine, sodium cholate, sodium acetate and optionally silver for use in the treatment of psoriasis, vitiligo and rosacea.

### TECHNICAL BACKGROUND

In the first 24 hours after injuries to the skin and mucosa, such as infections, bruises, haematomas, irritations, grazes, wounds and burns, an inflammatory reaction takes place. In skin lesions full of blood clots containing platelet aggregates, erythrocytes and leucocytes trapped in a fibrin lattice, blood vessel vasoconstriction factors are initially released to prevent further bleeding.

Subsequently, the increased blood supply due to vasodilation which carries leucocytes gives rise to subcutaneous oedema, high hydrostatic pressure, swelling, warmth and painful distension of the layer of epidermal cells or breakage of the skin.

If the skin is unbroken, blisters may form, as in the case of herpes zoster and burns, and inflammatory exudates can appear on the surface of the lesion.

Skin breakage can lead to major losses of fluid, and energy substrates (carbohydrates, lipids, proteins and biological effectors such as cytokines, trace elements, etc.) significantly alter the chemical composition of the internal environment at local level.

To ensure pain reduction and prevent a cell repair factor deficiency without a qualitative and quantitative variation in wound-healing, reducing the exudate must be viewed as a therapeutic act of primary importance. In this context, the treatment consists of antiseptic cleansing of the surface by means of a light compression bandage. The purpose of said compression is to stabilise and rapidly correct the inadequate nutrient composition in the skin lesion.

Psoriasis is an inflammatory disease of the skin, which is generally chronic. Although its etiology is not yet fully understood, it involves autoimmune, genetic, infectious and environmental factors (skin traumas or pharmacological treatments). Psoriasis commonly appears in the form of areas of thickened scaly skin, mainly located on the hands, feet, elbows, knees and scalp.

Therapy generally involves local treatment with steroids such as betamethasone, calcipotriol, keratolytic agents (salicylic acid, urea) or emollients (vaseline) to reduce the dryness of the skin, or tar. However, despite their efficacy, corticosteroids notoriously present a number of adverse effects. Tar must also be used with caution in view of its irritant action.

Vitiligo is a chronic skin disease characterised by the appearance of unpigmented blotches due to the absence of melanin. The etiopathogenesis of vitiligo is unknown, but autoimmune, genetic, metabolic and endocrine factors are believed to be involved. Vitiligo is often associated with serious autoimmune diseases, such as autoimmune disorders of the thyroid, alopecia areata, and naevi with a white halo (Sutton naevi). Neither systemic nor topical treatments are particularly effective in the long term, and must be repeated over time; they involve the use of immunosuppressants (such as tacrolimus or steroidal drugs), phototherapy, and the use of photosensitising substances such as psoralens.

Rosacea is a chronic form of dermatosis which is manifested by erythema, acneic lesions and telangiectasias, especially on the face. Once again its etiology is unclear, but the main factors involved are believed to be exposure to ultraviolet rays or infectious factors (intestinal Helicobacter pylori and mites at local level) that trigger an inflammatory response.

The treatment involves protection against sun exposure and the use of alcohol-free skin products, together with topical pharmacological treatment with metronidazole and azelaic acid. In more serious cases, systemic treatment with antibiotics is recommended.

French patent 2,850,276 describes a pharmaceutical composition containing betaine and steroidal surfactants for the treatment of dermatitis, mucosal lesions and slow-healing sores and ulcers. However, said composition is ineffective in the treatment of disorders such as psoriasis, vitiligo and rosacea.

### DESCRIPTION OF THE INVENTION

It has now been found that the combination of carnitine, sodium cholate, sodium acetate and optionally silver has a favourable action in the treatment of psoriasis, vitiligo and rosacea.

The present invention therefore relates to a composition comprising

a) carnitine,

b) sodium cholate,

c) sodium acetate,

and optionally

d) silver,

for use in the treatment of psoriasis, vitiligo and rosacea.

The effect of the compositions according to the present invention is greater than the sum of the effects obtained following separate administration of the individual ingredients of the combination. This superior effect is apparently due to synergy between the various ingredients.

The combination of the invention acts as a light compression bandage with an anti-exudative effect that accelerates wound healing.

The inclusion in the combination of the invention of a steroidal surfactant known to have a lipid-dissolving activity may seem paradoxical. However, in-depth studies have demonstrated that said ingredient, as well as performing an anti-infection activity, also aids the penetration of numerous medicaments.

It has been observed that topical administration of the composition in question to damaged tissues increases the subcutaneous concentrations of the surfactant components due to a synergic activity.

The composition of the invention will contain the active ingredients within the following weight ranges:

a) carnitine: 0.1 to 10.0 g,

b) sodium cholate: 0.1 to 10.0 g,

c) sodium acetate: 0.1 to 10.0 g,

and possibly

d) silver: 0.01 to 0.1 g.

The composition of the invention will preferably contain the active ingredients within the following weight ranges:

a) carnitine: 4.0 to 6.0 g,

b) sodium cholate: 4.0 to 6.0 g,

c) sodium acetate: 4.0 to 6.0 g,

and possibly

d) silver: 0.05 to 0.08 g.

The composition of the present invention can be formulated suitably for oral administration, and will be prepared by conventional methods well known in pharmaceutical technology, such as those described in Remington's Pharmaceutical Handbook, Mack Publishing Co., N.Y., USA.

Examples of formulations of the invention are set out below.

### Example 1—Gel Formulation

Gel Formula

| INCI description | % | Function |
|---|---|---|
| AQUA | <100 | Solvent |
| PVP | 12.00 | Film-forming/ |
| | | viscosity regulator |
| CARNITINE | 4.00 | Energy promoter/ |
| | | moisturising agent |
| SODIUM CHOLATE | 2.00 | Surfactant |
| HYDRATE | | |
| GLYCERIN | 2.00 | Moisturising agent |
| SODIUM ACETATE | 2.00 | Disinfectant/ |
| TRIHYDRATE | | preservative |
| XANTHAN GUM | 1.00 | Film-forming/ |
| | | viscosity regulator |
| PHENOXYETHANOL | 0.50 | Preservative |
| BENZYL ALCOHOL | 0.30 | Disinfectant/ |
| | | preservative |
| TOCOPHERYL ACETATE | 0.10 | Antioxidant |
| BENZALKONIUM | 0.10 | Disinfectant/ |
| CHLORIDE | | preservative |
| CI 77820 | 0.05 | Disinfectant/ |
| | | preservative |

### Example 2—Spray Formulation

Spray Formula

| INCI description | % | Function |
|---|---|---|
| AQUA | <100 | Solvent |
| CARNITINE | 4.00 | Energy promoter/ |
| | | moisturising agent |
| POLOXAMER 407-PPG12/ | 3.00 | Film-forming/ |
| SMDI COPOLYMER | | viscosity regulator |
| SODIUM ACETATE | 2.00 | Disinfectant/ |
| TRIHYDRATE | | preservative |
| SODIUM CHOLATE | 2.00 | Surfactant |
| HYDRATE | | |
| PHENOXYETHANOL | 0.60 | Preservative |
| BENZYL ALCOHOL | 0.30 | Disinfectant/ |
| | | preservative |
| BENZALKONIUM | 0.10 | Disinfectant/ |
| CHLORIDE | | preservative |

1. Composition comprising:
a) carnitine,
b) sodium cholate,
c) sodium acetate,
and optionally
d) silver
for use in the treatment of psoriasis, vitiligo and rosacea.

2. Composition according to claim 1, comprising the active ingredients within the following weight ranges:
a) carnitine: 0.1 to 10.0 g,
b) sodium cholate: 0.1 to 10.0 g,
c) sodium acetate: 0.1 to 10.0 g,
and optionally
d) silver: 0.01 to 0.1 g.

3. Composition according to claim 2, comprising the active ingredients within the following weight ranges:
a) carnitine: 4.0 to 6.0 g,
b) sodium cholate: 4.0 to 6.0 g,
c) sodium acetate: 4.0 to 6.0 g,
and optionally
d) silver: 0.05 to 0.08 g.
